SEQUENCE LISTING (17 sequences)

SEQ ID NO: 1 - protein, 4 aa, Homo sapiens
LNGA

SEQ ID NO: 2 - protein, 12 aa, Homo sapiens
AHCLEDAADGKV

SEQ ID NO: 3 - protein, 11 aa, Homo sapiens
AHSLSQPEPSK

SEQ ID NO: 4 - protein, 12 aa, Homo sapiens
HPDSEPDTIDHD

SEQ ID NO: 5 - protein, 5 aa, Homo sapiens
TRFGQ

SEQ ID NO: 6 - protein, 13 aa, Homo sapiens
AHCLEKSPRPSSY

SEQ ID NO: 7 - protein, 11 aa, Homo sapiens
AHQEVNLEPHV

SEQ ID NO: 8 - protein, 6 aa, Homo sapiens
EPTRKD

SEQ ID NO: 9 - DNA, 30 bp, Artificial Sequence
  Description of Artificial Sequence oligonucleotide primer
ctgaacggggcacacttctgtggaggcacc

SEQ ID NO: 10 - DNA, 33 bp, Artificial Sequence
  Description of Artificial Sequence oligonucleotide primer
tgccccgttcagtctaagactgacttgccaggg

SEQ ID NO: 11 - DNA, 43 bp, Artificial Sequence
  Description of Artificial Sequence oligonucleotide primer
ctggaggacgcagcagatggaaaggtaaaggtcatcctgggtg

SEQ ID NO: 12 - DNA, 42 bp, Artificial Sequence
  Description of Artificial Sequence oligonucleotide primer
tacctttccatctgctgcgtcctccaggcagtgggcagcagt

SEQ ID NO: 13 - DNA, 51 bp, Artificial Sequence
  Description of Artificial Sequence oligonucleotide primer
cacccagactcacagccagataccatcgatcacgatattgccttgctaaag

SEQ ID NO: 14 - DNA, 51 bp, Artificial Sequence
  Description of Artificial Sequence oligonucleotide primer
gtgatcgatggtatctggctgtgagtctgggtgcaagaacagcctagacac

SEQ ID NO: 15 - DNA, 46 bp, Artificial Sequence
  Description of Artificial Sequence oligonucleotide primer
ctttgatggctctggctgtgacagtgagtgtgcacccaggatgacc

SEQ ID NO: 16 - DNA, 51 bp, Artificial Sequence; identical to SEQ ID NO: 13
  Description of Artificial Sequence oligonucleotide primer

SEQ ID NO: 17 - protein, 810 aa, Homo sapiens
MEHKEVVLLLLLFLKSGQGEPLDDYVNTQGASLFSVTKKQLGAGSIEECAAKCEEDEEFTCRAFQYHSKEQQCVIMAENRKSSIIIRMRDVVLFEKKVYLSECKTGNGKNYRGTMSKTKNGITCQKWSSTSPHRPRFSPATHPSEGLEENYCRNPDNDPQGPWCYTTDPEKRYDYCDILECEEECMHCSGENYDGKISKTMSGLECQAWDSQSPHAHGYIPSKFPNKNLKKNYCRNPDRELRPWCFTTDPNKRWELCDIPRCTTPPPSSGPTYQCLKGTGENYRGNVAVTVSGHTCQHWSAQTPHTHNRTPENFPCKNLDENYCRNPDGKRAPWCHTTNSQVRWEYCKIPSCDSSPVSTEQLAPTAPPELTPVVQDCYHGDGQSYRGTSSTTTTGKKCQSWSSMTPHRHQKTPENYPNAGLTMNYCRNPDADKGPWCFTTDPSVRWEYCNLKKCSGTEASVVAPPPVVLLPDVETPSEEDCMFGNGKGYRGKRATTVTGTPCQDWAAQEPHRHSIFTPETNPRAGLEKNYCRNPDGDVGGPWCYTTNPRKLYDYCDVPQCAAPSFDCGKPQVEPKKCPGRVVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDSGGPLVCFEKDKYILQGVTSWGLGCARPNKPGVYVRVSRFVTWIEGVMRNN

(19) United States
(12) Patent Application Publication
(10) Pub. No.: US 2004/0235115 A1
(43) Pub. Date: Nov. 25, 2004

(54) Compositions and methods for treating thrombotic disorders

(76) Inventor: Guy L. Reed, Boston, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/715,981

(22) Filed: Nov. 18, 2003

Related U.S. Application Data
(60) Provisional application No. 60/427,152, filed on Nov. 18, 2002.

(51) Int. Cl. C07K 14/195; C12N 1/21; C07H 21/04

(52) U.S. Cl. 435/69.3; 435/320.1; 435/252.3; 530/324; 536/23.5

(57) ABSTRACT

The invention provides compositions containing a chimeric microplasmin polypeptide and methods for fibrinolytic therapy using the chimeric polypeptide.

### RELATED APPLICATIONS

This application claims priority to U.S. Provisional Application No. 60/427,152, filed on Nov. 18, 2002, the entire contents of which is hereby incorporated by reference.

### STATEMENT REGARDING FEDERAL FUNDING

This invention was made with U.S. Government support under National Institutes of Health grants HL64057. The government has certain rights in the invention.

### BACKGROUND OF THE INVENTION

The invention relates to blood clotting.

The serine protease plasmin cleaves fibrin to dissolve blood clots in a process know as fibrinolysis. Plasmin and its proenzyme plasminogen (Pg) also play important roles in cell migration, tissue remodeling, and bacterial invasion. The function of plasmin and Pg are regulated through protein interactions with plasminogen activators such as staphylokinase and streptokinase, substrates such as fibrin, inhibitors such as α2-antiplasmin, receptors, and other molecules. Glu-Pg, the physiological form of Pg, contains an amino-terminal segment, 5 kringle domains, and a protease domain. However, the role of structural elements in protein-protein interactions and substrate specificity is not well understood.

### SUMMARY OF THE INVENTION

The invention is based on elucidation of the role of structural elements (e.g., L3, L5, and L7 loops of microplasmin) in the interaction of microplasmin with its primary inhibitor α₂-antiplasmin. Accordingly, the invention provides a serine protease polypeptide such as plasmin (or microplasmin), the enzymatic activity of which is resistant to inhibition by a serpin such as α2-antiplasmin. For example, a chimeric microplasmin polypeptide contains a heterologous loop domain sequence, e.g., a loop sequence domain of a naturally-occurring factor D protein. The mutated microplasmin polypeptide contains at least 2, 4, 5, 8, 10, 12, 15, 20, 25 (and up to 100 or more) consecutive heterologous amino acids, e.g., factor D sequences.

The chimeric polypeptide is resistant to α2-antiplasmin inhibition compared to a wild type microplasmin. For example, the chimeric polypeptide is at least 10%, 20%, 50%, 75%, 100% more resistant to serpin inhibition. Preferably, the polypeptide is 2-fold, 5-fold, 10-fold more resistant. The level of fibrin cleavage activity is at least that of wild-type plasmin or exceeds that of the naturally-occurring protein (e.g., at least 1, 2, 5, 10, 25, 50% greater fibrinolytic activity compared to wild type plasmin).

The chimeric microplasmin polypeptide contains a heterologous loop domain sequence in microplasmin loop 3, 5, 6, or 7 of the microplasmin structure (FIG. 1). For example, the polypeptide contains amino acid sequence LNGA (SEQ ID NO:1) in microplasmin loop 3, amino acid sequence AHCLEDAADGKV (SEQ ID NO:2) in microplasmin loop 5, amino acid sequence AHSLSQPEPSK (SEQ ID NO:3) in microplasmin loop 6, and/or amino acid sequence HPDSEPDTIDHD (SEQ ID NO:4) in microplasmin loop 7. The microplasmin polypeptide lacks one or more of the following sequences: TRFGQ (SEQ ID NO:5) in microplasmin loop 3; AHCLEKSPRPSSY (SEQ ID NO:6) in microplasmin loop 5; AHQEVNLEPHV (SEQ ID NO;7) in microplasmin loop 6; and EPTRKD (SEQ ID NO:8) in microplasmin loop 7.

The compounds described herein are substantially pure. By a substantially pure polypeptide is meant a polypeptide, which is separated from those components (proteins and other naturally-occurring organic molecules) which naturally accompany it. A polypeptide is substantially pure when it constitutes at least 60%, by weight, of the protein in the preparation. Preferably, the protein in the preparation is at least 75%, more preferably at least 90%, and most preferably at least 99%, by weight, of the desired peptide. A substantially pure polypeptide is obtained, e.g., by extraction from a natural source; by expression of a recombinant nucleic acid; or by chemically synthesizing the protein. Purity is measured by a number appropriate methods known in the art, e.g., column chromatography, polyacrylamide gel electrophoresis, or HPLC analysis. A protein is substantially free of naturally associated components when it is separated from those contaminants which accompany it in its natural state. Thus, a protein which is chemically synthesized or produced in a cellular system different from the cell from which it naturally originates is substantially free from its naturally associated components.

In addition to peptides, the invention encompasses nucleic acids, e.g., oligonucleotides, which encode chimeric polypeptides. The nucleic acids, e.g., DNA or RNA, are substantially pure. By substantially pure DNA is meant DNA that is free of the genes which, in the naturally-occurring genome of the organism from which the DNA of the invention is derived, flank the desired gene sequence. The term therefore includes, for example, a recombinant DNA which is incorporated into a vector, into an autonomously replicating plasmid or virus, or into the genomic DNA of a prokaryote or eukaryote at a site other than its natural site; or which exists as a separate molecule (e.g., a cDNA or a genomic or cDNA fragment produced by PCR or restriction endonuclease digestion) independent of other sequences.

The peptides are prepared synthetically or by recombinant DNA technology. The term peptide is used interchangeably with polypeptide in the present specification to designate a series of amino acids connected one to the other by peptide bonds between the alpha-amino and alpha-carboxy groups of adjacent amino acids. Optionally, one or more peptide bonds are replaced with an alternative type of covalent bond (a “peptide mimetic”) which is not susceptible to cleavage by peptidases. Where proteolytic degradation of the peptides following injection into the subject is a problem, replacement of a particularly sensitive peptide bond with a noncleavable peptide mimetic yields a peptide mimetic, which is more stable and thus more useful as a therapeutic. Such mimetics, and methods of incorporating them into peptides, are well known in the art. Similarly, the replacement of an L-amino acid residue is a standard way of rendering the peptide less sensitive to proteolysis. Also useful are amino-terminal blocking groups such as t-butyloxycarbonyl, acetyl, theyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4,-dinitrophenyl. The polypeptides or peptides are either in their neutral (uncharged) forms or in forms which are salts, and either free of modifications such as glycosylation, side chain oxidation, or phosphorylation or containing these modifications, subject to the condition that the modification not destroy the immune stimulatory activity of the polypeptides.

Derivative peptide epitopes have an amino acid sequence, which differs from the amino acid sequence of a naturally-occurring receptor peptide. Such derivative peptides have at least 50% identity compared to a reference sequence of amino acids, e.g., a naturally-occurring glutamate receptor peptide. Preferably, a derivative is 90, 95, 98, or 99% identical to a naturally-occurring protein sequence. The derivative contains a conservative amino acid substitution. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu; Asp, Glu; Asn, Gln; Ser, Thr; Lys, Arg; and Phe, Tyr. Nucleotide and amino acid comparisons described herein are carried out using the Lasergene software package (DNASTAR, Inc., Madison, Wis.). The MegAlign module used is the Clustal V method (Higgins et al., 1989, CABIOS 5(2):151-153). The parameter used is gap penalty 10, gap length penalty 10.

The chimeric polyeptides are useful to dissolve blood clots, i.e., cleave fibrin clots. The invention therefore includes a method of dissolving a blood clot by contacting the blood clot with one or more of the chimeric polypeptides described above. For example, a subject, e.g., a human patient, suffering from or at risk of developing a thrombotic disorder is treated by administering to the subject one or more microplasmin-factor D chimeric polypeptides. Subjects to be treated include those who have experienced or are at risk of suffering from a heart attack, stroke, gangrene, deep venous thrombosis, peripheral arterial occlusion, or other disorder associated with an occlusion of a blood vessel. The chimeric polypeptides are administered to directly contact a blood clot, e.g., using a catheter to deliver the peptides to an anatomical site of an occlusion. The fibrinolytic agents of the invention have improved therapeutic value compared to wild-type plasmin, because the chimerics are more resistant to endogenous plasmin inhibitors, e.g., α2-antiplasmin, in blood.

Other embodiments and features of the invention will be apparent from the following description thereof, and from the claims.

### BRIEF DESCRIPTION OF THE DRAWINGS

FIG. 1 is a diagram showing a crystal structure of microplasmin. A ribbon diagram was created to illustrate those loops chosen for chimerization from PDB entry 1BML (Accession No. P00747) (Wang et al., 1998, Science 281:1662-1665). The side chains of the active site residues are shown. Sequence comparisons are shown between microplasminogen and factor D loops. This figure was produced using MOLSCRIPT and Raster3D (Kraulis et al., 1991, J. Appl. Crystallog. 24:946-950; Merritt et al., 1994, Acta. Crystallog. Sect. D 50:869-873).

FIG. 2A is a photograph of an electrophoretic gel showing purified microplasmin polypeptides. Purified recombinant wild-type or chimeric loop 3, 5, 6, and 7 microplasminogens (1 μM total protein, 75 μl) were cleaved with urokinase immobilized on agarose for 9 or 30 min. at 37° C. The microplasminogens were analyzed by SDS-PAGE on 10% gels under reducing conditions and stained with Coomassie blue dye. The relative migration of a 30 kDa standard is shown at left of the gel.

FIG. 2B is a bar graph showing activation human plasminogen by urokinase. Various microplasminogens (50 nM) were activated by 1 U urokinase and added to a solution of 0.5 mM S2251. Kinetics of activation were monitored at 405 nm over 20 minutes at 37° C.

FIG. 2C is a bar graph showing activation of human plasminogen by streptokinase. To test activation by streptokinase and S2251 was added to a final concentration of 0.5 mM S2551 to start the reaction. The 20 minute kinetics were measured at 405 nm absorbance.

FIG. 2D is a bar graph showing activation of human plasminogen by staphylokinase. Activation by staphylokinase was tested using 50 nM microplasmkinogens with excess staphylokinase and S2251 (0.5 mM). The kinetics were measured for 20 minutes at 405 nm.

FIGS. 3A and 3B are bar graphs showing binding of microplasminogens to streptokinase. The data shown in FIG. 3A depicts binding of an anti-plasminogen monoclonal antibody to immobilized wild-type and chimeric microplasminogens. The microplasminogens (25 μl, 20 μg/ml) or no protein (control) were absorbed on microtiter plates for 1 hr. The nonspecific protein binding was blocked by 1% BSA for 1 hr. After washing, an anti-plasminogen monoclonal antibody 340-11 was added for 1 hr. The bound antibody was detected by ¹²⁵I-(sheep antimouse Ab) (50,000 cpm) followed by gamma-counting. The data shown in FIG. 3B depicts binding microplasminogen chimeras to immobilized streptokinase. Streptokinase (10 ug/ml, 25 ul) was absorbed to the microtiter plate for 1 hr., blocked as before with 1% BSA, and the microplasminogens (25 μl., 20 μg/ml) or no microplasminogen (control) were added to the wells for 1 hr. The bound primary antibody was detected by ¹²⁵I-(sheep antimouse Ab (50,000 cpm) followed by gamma-counting.

FIG. 4. is a line graph showing inhibition of microplasmin or factor D by α₂ antiplasmin. Wild-type or chimeric microplasmins (10 nM) were added to cuvettes containing S-2251 (0.5 mM) in assay buffer and the change in absorbance at 405 nm recorded at 0.3 min. intervals prior and after the addition of human α₂ -antiplasmin (120 nM). The rate of loss of enzyme activity was determined and is expressed as log residual enzyme concentration versus time; the linear fits of the data are show. The interaction of factor D with α₂ -antiplasmin was determined using a standard assay for factor D activity (13). Factor D (20 nM final) and 0.13 mM DTNB final, were added to Tris buffer (100 mM, pH 8.0) and incubated at 37° C. for 3 minutes. Z-Lys-SBz1 substrate was added to start the reaction and the absorbance was measured at 412 nm at 0.3 min. intervals before and after the addition of human α₂ -antiplasmin. The rate of loss of the enzyme activity was then determined.

FIGS. 5A-D are graphs showing fibronolysis by chimeric microplasminogens. FIGS. 5A-B show fibronolysis with purified fibrin clots. Fibrinogen (5 μl, 20 mg/ml) and 3 μl ¹²⁵I-fibronogen were mixed with 37 μl TBS and CaCl₂ (2.5 μl, 0.4 M) and thrombin (2.5 μl, 1 unit/ml) were mixed and added to the fibrinogen solution to form the clot for 1 hr. at 37° C. After 5-6 hrs. incubation, the amount of residual clot was measured by gamma-counting and the fractional fibrinolysis was determined. In “pre-activation” experiments (FIG. 5A), 100 U of urokinase was added to the microplasminogens in assay buffer and incubated for 1 hr. at 37° C. The resulting microplasmins (200 μl) were then added as supernatant to the fibrin clots. In “synchronous” experiments (FIG. 5B), the mutant microplasminogens (0-1 μM) were added to the clots followed by the addition of 5 U urokinase. FIGS. 5C-D show fibrinolysis of human plasma clots in human plasma. For plasma clot lysis experiments, human plasma (100 μl) was mixed with trace amounts of ¹²⁵I-fibrinogen and clotted with CaCl₂ (20 nM) and thrombin (5 μl, 0.05 U) for 1 hr. at 37° C. The clots were suspended in 50 μl plasma as supernatant. In “pre-activation” experiments (FIG. 5C), 25 U of urokinase was added to the microplasminogens and incubated for 1 hr. at 37° C. The microplasmins (50 μl, 0-1 μM) were then added to the plasma supernatant. In “synchronous” experiments (FIG. 5D), the mutant microplasminogens (50 μl, 0-1 μM) were added to the clots followed by 5 U of urokinase.

### DETAILED DESCRIPTION

Clot lysis is mediated by plasmin in vivo. Under natural conditions, plasminogen is converted to plasmin by tissue plasminogen activator (t-PA) or other activator such as urokinase or streptokinase. After plasmin is set free into the circulation, it is rapidly combined with natural inhibitors.

Plasmin is the enzyme responsible for clot dissolution by cleaving fibrin in a process known as fibrinolysis. Studies were designed to investigate the role of structural elements in the interactions of the catalytic domain of plasmin (microplasmin) with major substrates and regulatory molecules. Factor D has no known endogenous inhibitors. Chimerization of wild-type microplasminogen was performed with complement Factor D such that the equivalent loop structure in Factor D replaced the native sequence in microplasminogen. Experimental results show that chimerization of L3, L5, and L7 1) does not affect protein expression or active site generation 2) has only modest effects on amidolysis 3) abolishes functional interactions with streptokinase 4) decreases the kinetics of inactivation of microplasmin by α₂-antiplasmin and 5) enhances the dissolution of human clots. Spectroscopic analysis confirms that Factor D is resistant to inhibition by α₂-antiplasmin. Chimerization of microplasminogen and Factor D leads to an increase in efficiency of fibrinolysis. The chimeric polypeptide therefore has improved therapeutic value in treating clotting disorders compared to native plasmin.

Fibrinolytic Activity of Plasmin

Plasminogen is the inactive precursor of the serine protease plasmin, and plasminogen activation is crucial in the fibrinolytic system that ultimately results in the dissolution of blood clots. The three-dimensional structure of microplasmin in complex with the bacterial plasminogen activators streptokinase and staphylokinase have revealed protein structures and intimate contacts required for plasminogen activation and interactions of plasmin with inhibitors. The two primary inhibitors of plasmin in the blood are α₂-macroglobulin and α₂-antiplasmin. Streptokinase and staphylokinase modify the ability of inhibitors to interact with plasmin. The staphylokinase-microplasmin complex retains its susceptibility to inhibition by α₂-antiplasmin. Conversely, the streptokinase-microplasmin complex is resistant to inhibition by α₂-antiplasmin.

Catalytically Efficient Plasminogen Fragments

A recombinant plasminogen fragment is more efficiently activated by a plasminogen activator (e.g., urokinase) in plasma and induces greater fibrinolysis compared to human glu-plasminogen. The fragments are characterized by a greater catalytic efficiency (Kcat/Kₘ) and/or faster kinetics (in terms of fibrinolysis) compared to human glu-plasminogen. The fragment contains at least 150 consecutive residues of SEQ ID NO:17. For example, the fragment contains at least 200 residues, or 225, 230, 235, 240, 245, 250, 255, 260 consecutive residues of SEQ ID NO:17. The fragment is less than 810 amino acids in length. Exemplary fragments contain residues 481-810, 550-810, 575-810, or 580-810 of SEQ ID NO: 17. The fragment optionally commences with an engineered methionine residue, i.e., the N-terminal residue of the fragment is a methionine residue. Mutated fragments include a polypeptide which spans residues 481-810, 550-810, 575-810, or 580-810 in which residues C555 and/or C560 are mutated, e.g., substituted with another residue such as alanine or serine; a polypeptide which spans residues 481-810, 550-810, 575-810, or 580-810 with residue R580 mutated to, e.g., subsituted with another residue such as alanine, serine, or glutamic acid. For example, cysteine residue is substituted with an acidic amino acid (eg., aspartic acid, glutamic acid), a basic amino acid (e.g., arginine, lysine, histidine), a hydroxyl amino acid (e.g., serine or threonine), or a hydrophobic residue (e.g., alanine, valine, leucine, or isoleucine). For example, the arginine residue is substituted with an acidic amino acid (eg., aspartic acid, glutamic acid), another basic amino acid (e.g., lysine, histidine), a hydroxyl amino acid (e.g., serine or threonine), or a hydrophobic residue (e.g., alanine, valine, leucine, or isoleucine). Preferably, the fragments are shorter in length than a naturally-occurring proteolytic fragment of plasminogen, e.g., a fragment containing residues 481-810, residues 452-810, or residues 445-810, of SEQ ID NO:17. The fragments are at least 10%, and preferably 20%, 50%, 100% and up to 2-fold, 5-fold, 10-fold more catalytically efficient compared to glu-plasminogen. These polypeptides or plasminogen activators lyse clots with greater efficiency compared to native plasminogen proteins. Activation of plasminogen, kinetics of catalytic activation, and fibrinolysis are measured using methods well known in the art.

TABLE 3

Human plasminogen precursor
(SEQ ID NO: 17; GENBANK ™ Accession No. P00747)

| | |
|---|---|
| 1 | mehkevvlll llflksgqge plddyvntqg aslfsvtkkq lgagsieeca akceedeeft |
| 61 | crafqyhske qqcvimaenr kssiiirmrd vvlfekkvyl secktgngkn yrgtmsktkn |
| 121 | gitcqkwsst sphrprfspa thpsegleen ycrnpdndpq gpwcyttdpe krydycdile |
| 181 | ceeecmhcsg enydgkiskt msglecqawd sqsphahgyi pskfpnknlk knycrnpdre |
| 241 | lrpwcfttdp nkrwelcdip rcttpppssg ptyqclkgtg enyrgnvavt vsghtcqhws |
| 301 | aqtphthnrt penfpcknld enycrnpdgk rapwchttns qvrweyckip scdsspvste |
| 361 | qlaptappel tpvvqdcyhg dgqsyrgtss ttttgkkcqs wssmtphrhq ktpenypnag |
| 421 | ltmnycrnpd adkgpwcftt dpsvrweycn lkkcsgteas vvapppvvll pdvetpseed |
| 481 | cmfgngkgyr gkrattvtgt pcqdwaaqep hrhsiftpet npraglekny crnpdgdvgg |
| 541 | pwcyttnprk lydycdvpqc aapsfdcgkp qvepkkcpgr vvggcvahph swpwqvslrt |
| 601 | rfgmhfcggt lispewvlta ahcleksprp ssykvilgah qevnlephvq eievsrlfle |
| 661 | ptrkdiallk lsspavitdk vipaclpspn yvvadrtecf itgwgetqgt fgagllkeaq |
| 721 | lpvienkvcn ryeflngrvq stelcaghla ggtdscqgds ggplvcfekd kyilqgvtsw |
| 781 | glgcarpnkp gvyvrvsrfv twiegvmrnn |

Complement Factor D

Complement factor D is a serine protease in the serum that is essential in the formation of the C3 convertase of the alternative pathway of complement activation. Factor D circulates in the blood as an intrinsically active serine protease and no known endogenous inhibitor has been identified, serpin-like or otherwise. A unique “self-inhibitory” loop of factor D modulates the self-regulation and high specificity of the enzyme. The need for a serpin-like or other endogenous inhibitor is believed to be precluded by the action of two molecular processes: (1) the “self-inhibitory” loop and (2) a conformational change upon binding of factor D to C3b-bound factor B that results in expression of proteolytic activity. The three-dimensional structures of plasminogen and factor D have a conserved structure that is similar to other serine proteases such as trypsin; plasminogen and factor D have an RMSD of 1.06. The differences in the structures are primarily in the loop regions of the enzymes. The length of the loops, due to insertions or deletions and the conformation of the loops may vary among the serine proteases. The variation of the loops and the otherwise high conservation in three-dimensional structure, suggest that the loops are important in mediating interactions with substrates and inhibitors.

Chimeric Polypeptides

Inhibition of the bacterial plasminogen activator and plasmin complexes by α₂-antiplasmin combined with the respective X-ray crystallographic structures of the complexes suggest the involvement of loops 3, 5, and 7 in the interaction of plasmin with α₂-antiplasmin. To investigate microplasmin inhibition by α₂-antiplasmin three mutations were created using overlap PCR. The sequences of loops 3, 5, and 7 of factor D were used to replace the native sequence of the equivalent loop in microplasminogen. The effect of these loop mutations on the interaction of microplasmin with its substrates was then assayed.

Cloning, Purification, & Expression of Mutant Proteins

Site-directed mutagenesis of the microplasminogen sequence was performed with polymerase chain reaction with overlap extension using the following primers: L3 sense, CTGAACGGGGCACACTTCTGTGGAGGCACC (SEQ ID NO:9), and L3 antisense TGCCCCGTTCA GTCTAAGACTGACTTGCCAGGG (SEQ ID NO:10); L5 sense, CTGGAGGACGCAGCAGATGGAAA GGTAAAGGTCATCCTGGGTG (SEQ ID NO:11), and L5 antisense, TACCTTTCCATCTGCTGCGTCC TCCAGGCAGTGGGCAGCAGT (SEQ ID NO:12); L7 sense, CACCCAGACTCACAGCCAGATACCA TCGATCACGATATTGCCTTGCTAAAG (SEQ ID NO:13), and L7 antisense, GTGATCGATGGTATC TGGCTGTGAGTCTGGGTGCAAGAACAGCCTAGACAC (SEQ ID NO:14). The amino acid sequence and nucleotide sequence of wild type microplasminogen is known in the art and accessible to those skilled in the art.

The PCR products were sequenced on both strands to confirm the target mutations and the recombinant microplasminogens were ligated into pET11d for bacterial expression. Recombinant microplasminogens were purified from inclusion bodies and refolded in 55 mM Tris pH 8.2, with 10.56 mM NaCl, 0.44 mM KCl, 0.055% polyethylene glycol 3350, 2.2 mM MgCl₂, 2.2 mM CaCl₂, 550 mM arginine, 1 mM reduced glutathione, 0.1 mM oxidized glutathione. Purified proteins were then dialyzed in 100 mM Tris pH 8.0 and 10 mM EDTA. The strategy of loop chimerization is summarized in Table 1.

TABLE 1
Strategy of Loop Chimerization

| | | |
|---|---|---|
| Pg loop No./ | Factor D | |
| Sequence | Sequence | Primers |

| | | | |
|---|---|---|---|
| 3 | TRFGQ | LNGA | CTGAACGGGGCACTTCTGGAGGCACC |
| | (SEQ ID NO:5) | (SEQ ID NO:1) | |
| | | | TGCCCCGTTCAGTCTAAGACTGACTTGCCAGGG |
| | | | (ID NO:9) |
| 5 | AHCLEKSPRPSSY | AHCLEDAADGKV | CTGGAGGACGCAGCAGATGGAAAGGTAAAGGT |
| | (SEQ ID NO:6) | (SEQ ID NO:2) | CATCCTGGGTG (SEQ ID NO:10) |
| | | | TACCTTTCCATCTGCTGCGTCCTCCAGGCAGTG |
| | | | GGCAGCAGT (SEQ ID NO:11) |
| 6 | AHQEVNLEPHV | AHSLSQPEPSK | TCACTGTCACAGCCAGAGCCATCAAAGCAGGA |
| | (SEQ ID NO:7) | (SEQ ID NO:3) | AATAGAAAGTGTCTAGG (SEQ ID NO:12) |
| | | | CTTTGATGGCTCTGGCTGTGACAGTGAGTGTGC |
| | | | ACCCAGGATGACC (SEQ ID NO:15) |
| 7 | EPTRKD | HPDSQPDTIDHD | CACCCAGACTCACAGCCAGATACCATCGATCAC |
| | (SEQ ID NO:8 | (SEQ ID NO:4) | GATATTGCCTTGCTAAAG (SEQ ID NO:16) |
| | | | GTGATCGATGGTATCTGGCTGTGAGTCTGGGTG |
| | | | (SEQ ID NO:13) |
| | | | CAAGAACAGCCTAGACAC (SEQ ID NO:14) |

Sense primers are listed first followed by antisense primers.

Active Site Titration

The molar quantities of the active sites generated by the recombinant microplasmins was determined at 37° C. in a Hitachi 2500 fluorescence spectrophotometer by active site titration with the fluorogenic substrate 4-methylumbelliferyl p-guanidinobenzoate (Sigma) using know methods.

Kinetic Assays of Microplasminogen Recombinants Amidolysis

The amidase kinetic parameters of the microplasmins were determined by measuring the cleavage of the paranitroanilide substrate S2251 (H-D-valyl-L-leucyl-L-lysine-p-nitroanilide dihydrochloride, Chromogenix, Sweden). Microplasmins (20 nM) and assay buffer (50 mM Tris-HCl, 100 mM NaCl, pH 7.4) were in a quartz cuvette at 37° C. and various concentrations of S2251 (0.50-4.0 mM) were added to obtain a final volume of 300 μl. The change in absorbance was measured at 405 nm for 5 min at 37° C. in a thermostatted Cary 100-Bio spectrophotometer. The data were plotted as V/S and analyzed by hyperbolic curve fitting using a Sigma Plot program.

Plasminogen Activation

Microplasminogens (100 μl, 1 μM) were mixed with urokinase (5 U) or streptokinase (100 nM) and 40 μl of this solution was added to microtiter plate wells containing 20 μl S-2251 and 140 μl assay buffer. The conversion of plasminogen to plasmin was detected by the cleavage of S2251 by monitoring the rate of change in A405 over time.

Inhibition of Microplasmin Mutants by α₂-Antiplasmin

Wildtype or mutant microplasmins (10 nM) were added to cuvettes containing S2251 (500 nM) in assay buffer and the change in absorbance at 405 nm recorded for 60 sec prior to the addition of human α₂-antiplasmin (120 nM, from Calbiochem). The rate of loss of enzyme activity was determined and is expressed as residual enzyme concentration vs. time.

Plasma Clot Lysis by Mutant Microplasminogens

Packed, fresh frozen human plasma (100 μl) was mixed with trace amounts of ¹²⁵I-fibrinogen and clotted with CaCl₂ (20 mM) and thrombin (5 μl, 0.05 U) for 1 hr. at 37° C. After washing in 1 mL of Tris-buffered saline with 10 μM P-PACK (to inhibit residual thrombin), the clots were suspended in 50 μl plasma (10 μM P-PACK). The mutant microplasminogens (50 μl, 0-1 μM) were added to the clots followed by 5 U of urokinase. After incubation overnight the amount of the residual clot was measured by gamma-counting and the fractional fibrinolysis was determined using standard methods.

Fibrinogen Clot Lysis Assay

Fibrinogen (5 μl, 20 mg/ml from American Diagnostica) was mixed with trace amounts of ¹²⁵I-fibrinogen, and clotted by the addition of CaCl₂ (2.5 μl, 0.4 M) and thrombin (2.5 μl) for 1 hr at 37° C. The mutant microplasminogens (0-1 μM) were added to the clots followed by the addition of 5 U of urokinase to activate the microplasminogens. The residual clot was measured at 0, 1, and 2 hrs by gamma-counting and the fractional fibrinolysis was determined.

Interaction of Factor D and α₂-Antiplasmin

To assay the interaction of Factor D with α2-antiplasmin 250 μl 0.10 M Tris buffer, pH 8.0, 30 μl of 200 nM Factor D, and 3 μl of 0.13 M 5,5′-dithiobis (2-nitrobenzoic acid) (DTNB) were incubated at 37° C. for 3 minutes. Then 15 μl of Z-Lys-SBz1 (Enzyme Systems Products, USA) was added to 20 mM final concentration to start the reaction and the absorbance was measured at 412 nm over 4-5 minutes. After the reaction was allowed to proceed α2-antiplasmin was added to a final concentration of 100 nM and the reaction mixture continually monitored at 412 nm.

Binding Assays

The binding of microplasminogens to streptokinas was studied in microtiter plates. The microplasminogens (25 μl, 20 μg/ml) or no protein (control) were adsorbed on microtiter plates for 1 hour. Non-specific protein binding was blocked with 1% bovine serum albumen for 1 hour. After washing, an anti-plasminogen monoclonal antibody (e.g., Ab 340-11) was added for 1 hour. The amount of bound antibody was measured using a detectable label such as ¹²⁵I-(sheep anti-mouse Ab; 50,000 cpm) followed by gamma-counting.

The binding of microplasminogens to streptokinase was also studied in a microtiter plate. Steptokinase (10 μg/ml, 25 μl) was adsorbed to the microtiter plates for 1 hour, blocked with 1% BSA, and microplasminogens (25 μl, 20 μg/ml) or no microplasminogen (control) were added to the wells for 1 hour. The bound primary antibody was detected as described above.

Chimerization of Microplasminogen

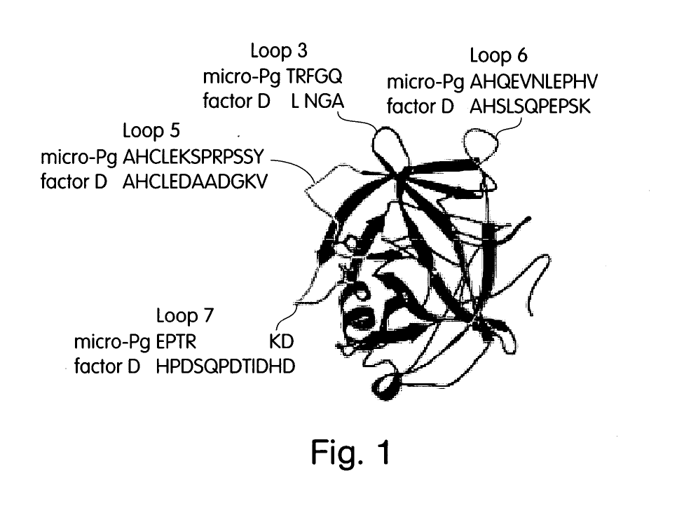

Crystal structure data indicates that streptokinase interacts with microplasmin at loops 3, 5, 6 and 7, while staphylokinase interacts only with loop 7. Through these interactions streptokinase renders plasmin unable to cleave fibrin and resistant to inhibition by α2-antiplasmin. Corresponding to the functional differences between microplasmin and factor D, there are notable differences in the sequences of these loops between these two molecules (FIG. 1). For example loop 7 of factor D is longer than loop 7 of microplasmin and shows a different electrostatic potential. Negatively charged clusters are seen in factor D and positively charged clusters seen in microplasminogen. Loop 3 of factor D lacks negatively charged residues while microplasminogen has an arginine present. Loop 5 of factor D has similarities with microplasminogen with the notable difference in overall charge (positive vs. negative, respectively). Loop 6 of factor D is also similar to microplasmin though the overall charge of factor D is different (positive). To examine the contribution of these structures to mediating interactions of microplasmin, loop chimeras were created. The chimeras were expressed in bacteria, purified and refolded to obtain active protein.

Interaction of Chimeric Microplasminogens with Plasminogen Activators

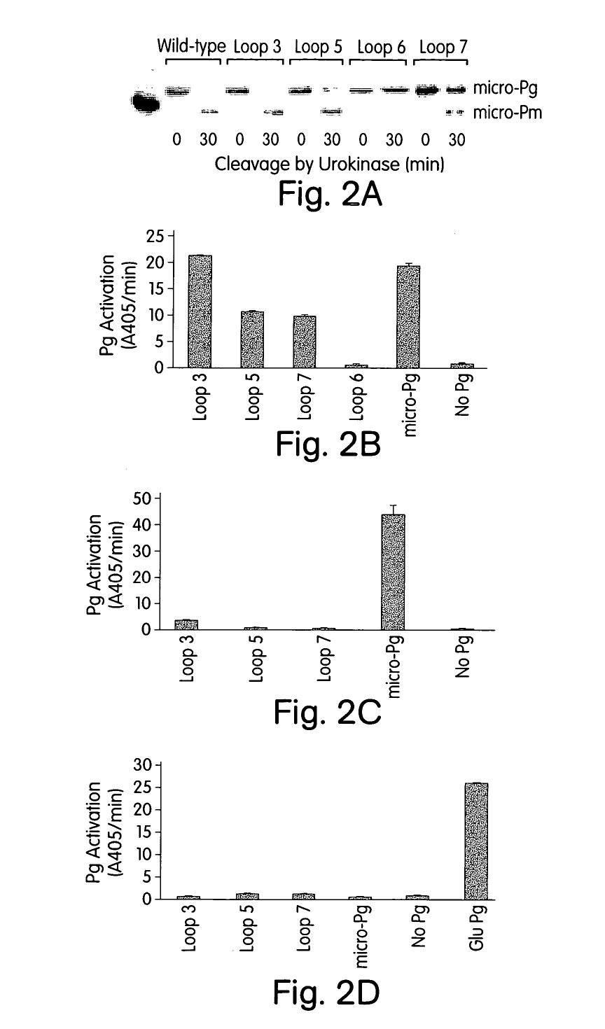

The plasminogen activator urokinase is a serine protease that directly cleaves plasminogen to create plasmin. Each microplasminogen was treated with various amounts of urokinase for different lengths of time to determine the amount of active protein by active site titration with the fluorogenic substrate MUGB. Wild-type microplasminogen and the loop 3 and 5 chimeric microplasminogens were more readily cleaved by urokinase than was the loop 7 chimera (FIG. 2A). Following cleavage by urokinase, wild-type microplasminogen and the loop 3, 5, and 7 chimeric microplasminogens developed amidolytic activity with a plasmin-selective substrate S225 I (FIG. 2B). In contrast, the loop 6 chimera was not cleaved by urokinase (FIG. 2A) even after prolonged incubation and never developed proteolytic activity (FIG. 2B). These data suggested that it may not be refolded to active protein.

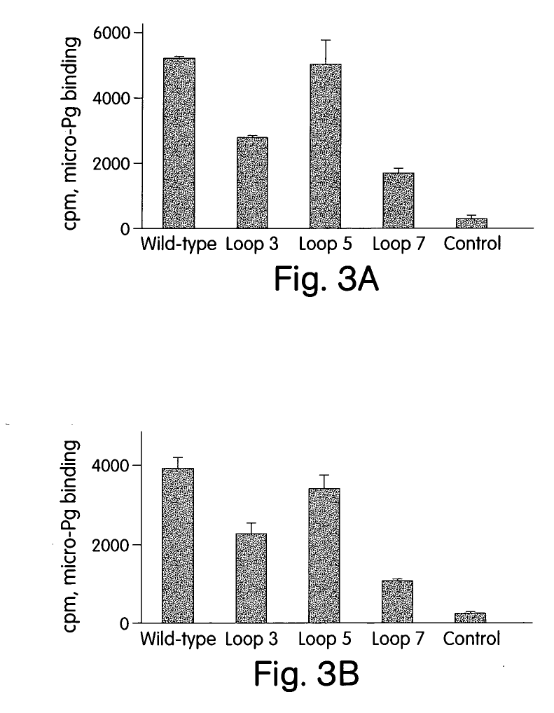

Molecular modeling of the crystal structure predicted that loop 3, loop 5 and loop 7 of microplasmin are involved in interactions with streptokinase in the activator complex. Consistent with these observations all three loop mutants were not efficiently activated by the bacterial plasminogen activator streptokinase (FIG. 2C). Because complex formation between microplasminogen is required for activation by streptokinase, the binding of streptokinase to these domains was examined with a monoclonal antibody directed to the protease domain (eg., MAb 340-11). When compared to wells containing no microplasminogen, MAb 340-11 showed specific binding to each chimeric microplasminogen (FIG. 3A), although it bound with less avidity to the loop 3 and 7 chimeras. This permitted the use of the MAb to examine microplasminogen binding to immobilized streptokinase in a similar assay. When compared to control (no microplasminogen) all microplasminogen chimeras bound to streptokinase (FIG. 3B) in a pattern that simulated the binding of microplasminogen alone (FIG. 3A). These data indicated that the failure of streptokinase to activate these microplasminogen chimeras was not simply due to an inability of these proteins to bind to each other.

The ability of staphylokinase to activate the microplasminogen chimeras was tested to further elucidate the interaction of these loops with plasminogen activators. Unlike urokinase (FIG. 2B) or streptokinase (FIG. 2C), staphylokinase was unable to efficiently activate the wildtype microplasminogen (FIG. 2D). Staphylokinase was unable to activate the chimeric microplasminogens though it was able to efficiently activate Glu-plasminogen (FIG. 2D). This result indicates the importance of kringle domains in the staphylokinase mechanism of activation.

Activation of Microplasminogens

All three of the microplasminogen recombinants failed to be activated by the bacterial plasminogen activator streptokinase. Proteolytic cleavage by urokinase resulted in variable activation of the mutants. The L3 microplasminogen mutant resulted in a greater than 50% decrease in activation in comparison to wildtype. L5 and L7 mutants showed about 80% decrease in activity compared to wildtype microplasminogen. Molecular modeling of the crystal structure of the catalytic domain of human plasmin complexed with streptokinase reveals involvement of an arginine in L3 that mediates important interactions with streptokinase. An equivalent residue is missing in the Factor D L3 sequence insertion thus abolishing the potential interaction. L5 and L7 are involved in multiple interactions with the beta and alpha domains of streptokinase, respectively, and mutation of these loop residues may eliminate important stabilizing interactions for streptokinase binding and thus affect microplasminogen activation.

Wildtype microplasminogen and the L5 and L3 mutants resulted in similar generation of active sites at the end of a 1 hr. incubation, while L7 showed decreased active site generation. There were modest changes in the kinetic parameters of the mutant microplasmins when compared to wildtype microplasmin. The L3 recombinant retained kinetic activity similar to wildtype microplasmin while the L5 and L7 recombinants had the greatest decrease in catalytic efficiency. Mutation of L7 was the most radical change from the native microplasminogen, involving the insertion of 6 additional residues in the protein sequence, thus enlarging the loop's size and perhaps altering its stability in solution. Although, some researchers have suggested that the three proline residues at positions 90, 92, and 96 of Factor D likely provide an internal rigidity to L7, also contributing to the decrease in catalytic activity may be the introduction of Factor D's serine at position 94 which is a bulky group in other trypsin-like serine proteases. The presence of a bulky group precludes an atypical His 57 confirmation and prevents a decrease in enzyme activity.

Effect of Chimerization on Amidolysis Kinetics

Chimerization had modest effects on the kinetic parameters of the mutant microplasmins when compared to wild-type microplasmin (Table 2). Chimerization of loop 3 did not significantly alter the kinetics of amidolysis compared with wild-type microplasmin. Chimerization of loop 7 slightly reduced catalytic efficiency by approximately 2-fold. The loop 5 chimera displayed an increased Km (approximately 3-fold) and a decreased catalytic efficiency (approximately 2-fold).

TABLE 2
Kinetic Parameters for Amidolysis

| Enzyme | | Amidolytic Parameters | |
|---|---|---|---|
| Microplasmin type | Kₘ(μM) | k_{cat}(s⁻¹) | k_{cat}/Kₘ(μM⁻¹s⁻¹) |
| wild-type | 2030 ± 284 | 20.5 ± 1.5 | 0.010 |
| loop 3 | 2920 ± 850 | 35.2 ± 5.1 | 0.012 |
| loop 5 | 5980 ± 852 | 27.0 ± 2.5 | 0.005 |
| loop 7 | 3310 ± 705 | 17.5 ± 3.7 | 0.005 |

Experiments were carried out as 37° C. in total volume of 300 μl and kinetic parameters were determined as described in Experimental Procedures. The values represent the mean ±SE.

Inhibition by α2-Antiplasmin

The mutants show differing resistance to α₂-antiplasmin in comparison to the wildtype microplasmin as illustrated. The L7 and L5 mutants are similarly affected by the plasmin inhibitor, and are 3 times and 2.5 times more resistance to inhibition, respectively, when compared to wildtype. The L3 mutant is approximately 1.5 times more resistant to inhibition than wildtype.

The reaction between plasmin and antiplasmin proceeds via two steps: 1) a very fast reversible second-order reaction and 2) a slower irreversible first-order reaction. In the experiments described herein, the catalytic subunit of plasmin was used which is lacking the kringle domains that are largely responsible for the fast reaction rate. The inhibition experiments reveal the role that the loops play in mediating the interaction of the catalytic subunit with α₂-antiplasmin. Mutation of the native loops of microplasmin to the sequence of the equivalent loop of Factor D results in resistance to inhibition either by the loss of critical residues needed for recognition by α₂-antiplasmin and/or residues are introduced that assist in repulsion of the plasmin inhibitor (i.e. charge reversal, steric factors).

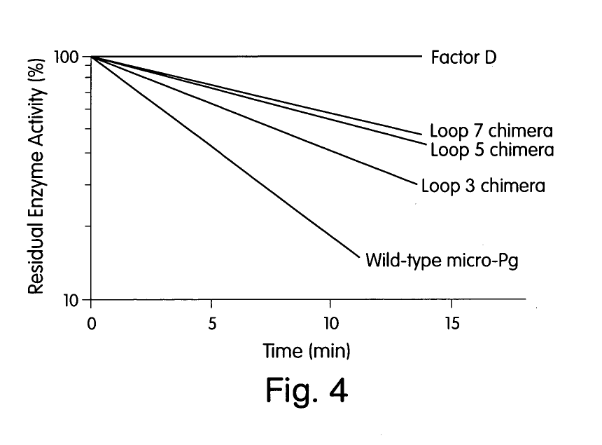

Factor D was found to be resistant to the inhibitory effects of the serine protease inhibitor α2-antiplasmin while wild-type microplasmin was rapidly inhibited (FIG. 4, k₁=19900±430 M⁻¹s⁻¹). Chimerization with factor D loops altered the resistance of microplasmin to α2-antiplasmin. The loop 5 (k₁=7780±270 M⁻¹s⁻¹) and loop 7 (k₁=6131±440 M⁻¹s⁻¹) chimeras were 2-3 fold more resistant to inhibition than wild-type microplasminogen. The loop 3 mutant also displayed reduced kinetics of inhibition (k₁=14800±900 M⁻¹s⁻¹) when compared to wild-type microplasminogen.

Effects of Chimerization on Fibrin and Plasma Clot Lysis

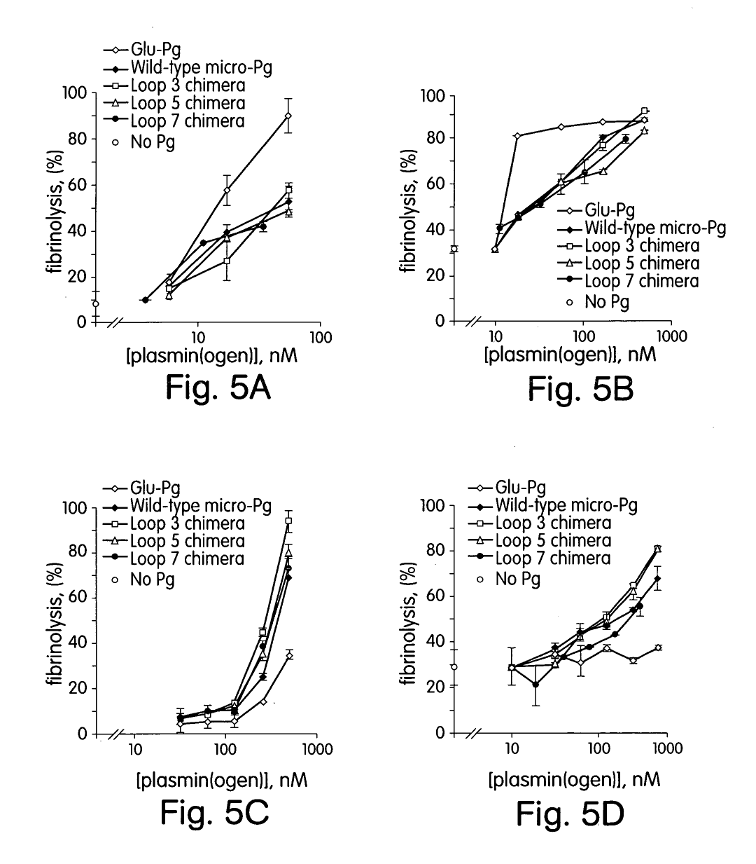

Experiments were performed to investigate the effect of chimerization on the interactions of microplasmin with fibrin, its chief physiologic substrate. When microplasminogens were “pre-activated” to plasmin and added to fibrin clots in the absence of inhibitor, plasmin was more effective in fibrinolysis than wild-type microplasmin. However, there was little or no discernible difference between wild-type and chimeric microplasmins in the efficiency of fibrinolysis. When Glu-plasminogen and the microplasminogens were added to fibrin clots with urokinase, Glu-plasminogen was the most potent fibrinolytic agent and the chimeras were similar to wild-type microplasminogen in their ability to cleave fibrin (FIG. 5B). Thus, introduction of the loop mutations did not alter the ability of the chimeras to cleave fibrin.

Normally fibrinolysis occurs in plasma or blood which contains Glu-plasminogen, α2-antiplasmin and other inhibitors of plasmin. Tthe effect of chimerization on fibrinolysis of human clots in human plasma was evaluated. When Glu-plasminogen and the recombinant microplasminogens were pre-activated with urokinase and added to human plasma clots the pattern of fibrinolysis (FIG. 5C) was opposite to that seen with fibrin clots (FIG. 5A). Recombinant wild-type microplasminogen was approximately 2-fold more potent than Gluplasminogen in achieving equivalent lysis (FIG. 5C). The loop 3, loop 5 and loop 7 chimeras were up to 3-fold more potent than Glu-plasminogen and up to 1.5-fold more potent than wild-type microplasminogen. When the different plasminogens were added to plasma with urokinase a similar pattern was seen but the differences between the microplasminogens (wild-type and mutants) and Glu-plasminogen were more marked (5-6 fold, FIG. 5D).

Effect of α₂-Antiplasmin on Factor D Activity

Colorimetric analysis revealed that α₂-antiplasmin had no effect on the rate of esterolysis of the substrate. Factor D is resistant to the inhibitory effects of the serine protease inhibitor α₂-antiplasmin. These studies assist in confirming statements that there is no known endogenous inhibitor of Factor D. It is likely that a unique self-inhibitory loop did away with the necessity of Factor D being regulated as a zymogen or by other serpin-like inhibitors in the blood. The activity of Factor D is regulated by induction of an active-state conformation from a resting-state conformation by C3b-bound factor B.

Protein Interactions of Loop Domains of Plasmin and Chimeric Microplasminogen Polypeptides

The data described herein indicate that the differences in biological activity between plasmin and factor D are due to sequence divergences in the flexible loops of the catalytic domains which target the function of these proteases to specific sites and substrates. Studies were undertaken to understand the contribution of these structural elements to the interactions of the protease domain of plasmin with streptokinase, fibrin and α-antiplasmin. Loops 3, 5, 6 and 7 of microplasminogen are shielded from solution when plasmin forms a complex with streptokinase while only loop 7 is shielded in the staphylokinase-microplasmin complex. The fact that streptokinase, but not staphylokinase modifies the ability of plasmin to interact with fibrin and α-antiplasmin indicated that these loops participate in substrate and inhibitor interactions.

As described above, plasmin is inhibited by α-antiplasmin in two steps: 1) a very fast reversible second order reaction and 2) a slower irreversible first-order reaction. The first step in this reaction involves interactions between the kringle domains of plasmin and the carboxy terminus of α2-antiplasmin. The results described herein indicated that chimerization of loops 5 and 7, and to a lesser extent loop 3, reduced the ability of α-antiplasmin to inhibit microplasmin. This provided the first experimental evidence that structural elements in the protease domain are important for the interactions of these two molecules. These loop residues are involved in the proper alignment and positioning of α2-antiplasmin with respect to the catalytic triad. Chimerization altered intermolecular contacts between plasmin-α2-antiplasmin by deleting critical residues, altering residue charge or creating steric interference. Chimerization of loop 3 had only mild effects on reducing the inhibition of microplasmin by α2-antiplasmin, suggesting that this is not a major site of interaction between these two molecules. Microplasminogen chimeras gained resistance to α2-antiplasmin inhibition through disruption of residues required by microplasmin for recognition of α2-antiplasmin.

The data described herein provide biochemical evidence for the role of loops 3, 5 and 7 in activator complex formation with streptokinase. Although streptokinase formed a complex with all three chimeras (FIGS. 3A-B), it was unable to generate an active site in plasminogen. In contrast, another plasminogen activator, urokinase, cleaved each of the loop chimeras to plasmin. The effects of the loop 3 chimera in disrupting interactions with streptokinase are particularly notable since this represented a comparatively conservative change in microplasmin with deletion of Arg 582 and substitution of homologous residues. In the loop 3 chimera, Thr 581 was mutated to Leu. Moreover, in the structure of the microplasmin with streptokinase, loop 3 interacts with the beta domain which, compared with the alpha and gamma domains of streptokinase has comparatively few intermolecular contacts. Deletion of Thr 581 in loop 3 also caused a loss of streptokinase-microplasmin complex activity. In the streptokinase-microplasmin structure loop 5 also contacts the beta domain and molecular modeling suggests that within this loop Arg 610 has important interactions with streptokinase. Finally, chimerization introduces a large insertion into loop 7 so that steric factors along with an increase in negatively charged residues contribute to abolition of the ability to form a functional activator complex.

The role of the loop mutations in forming an activator complex with staphylokinase could not be evaluated under the conditions of these experiments because staphylokinase, though it efficiently activated Glu-Pg (the physiologic form of Pg), showed no significant ability to activate microplasminogen (even after 8 hrs of incubation, FIG. 2D). Molecular modeling indicates that loop chimeras 3 and 5 would be similar to wild-type microplasmin in their ability to form a Pg activator as these loops are not in contact with the staphylokinase molecule in the activator complex.

Chimerization did not significantly affect the ability of microplasmin to cleave fibrin. Wild-type and chimeric microplasminogens had equivalent fibrinolytic effects, either when added with urokinase to fibrin clots or when pre-activated by urokinase and then added to fibrin clots. In the same experiments, greater fibrinolysis was obtained with Glu-Pg and urokinase; this probably reflects enhanced fibrin-targeting by the kringle domains. However, when fibrinolytic experiments were performed in the presence of plasma, which contains α2-antiplasmin and other inhibitors, an opposite result was obtained. Wild-type and chimeric microplasminogens were significantly more effective than Glu-plasminogen as fibrinolytic agents, particularly when the plasminogens and urokinase were added to plasma together. This probably reflects the slower inhibition by; -antiplasmin of microplasmin which lacks kringle domains. The fact that microplasmin(ogen) was more potent than Glu-plasmin(ogen) when activated in plasma (FIG. 5D), than when pre-activated and added to plasma, (FIG. 5C) suggests that microplasminogen may be a more efficient substrate for urokinase than Glu-Pg. The increased potency of the chimeric microplasminogens was due at least in part to the enhanced resistance of these chimeras to α2-antiplasmin. Loop 3 chimera has superior potency but displayed less resistance to α2-antiplasmin than the other chimeras. Other factors such as differential rates of plasminogen activation, variable resistance to α2-macroglobulin, enhanced stability in plasma, etc., may play a role in affecting fibrinolysis by these molecules.

Loop structures in the protease domain were found to specifically modulate the functional interactions of microplasmin(ogen) with certain regulatory molecules and inhibitory molecules. The microplasmin-factor D hybrid polypeptides described herein have numerous advantages over naturally-occurring plasmin for treating thrombotic disorders, e.g., increased resistance to endogenous inhibitors and increased efficiency in dissolving blood clots.

Therapeutic Administration of Chimeric Polypeptides

The peptides are administered at an intravenous dosage of approximately 1 to 100 moles of the polypeptide per kg of body weight per day. The compositions of the invention are useful for parenteral administration, such as intravenous, subcutaneous, intramuscular, intraperitoneal. The chimeric microplasmin polypeptides are delivered to a site of a thrombus using a catheter.

A unit dose of the peptide ranges from 0.1 to 100 mg, which may be administered at one time or repeatedly to a patient. A plurality of peptides are optionally administered is together (simultaneously or sequentially).

Peptides are recombinantly produced or synthetically made using known methods. Peptide solutions are optionally lyophilized or granulated with a vehicle such as sugar. When the compositions are administered by injection, they are dissolved in distilled water or another pharmaceutically acceptable excipient prior to the injection.

DNA encoding a chimeric peptide may also be administered, e.g., by incorporating the DNA into a viral vector. Nucleic acids are adminstered using known methods, e.g., intravenously, at a dose of approximately 10⁶ to 10²² copies of the nucleic acid molecule.

Other embodiments are within the following claims.

What is claimed is:

1. A microplasmin polypeptide comprising a heterologous loop domain sequence, wherein said polypeptide is resistant to α2-antiplasmin inhibition compared to a wild type microplasmin.

2. The polypeptide of claim 1, wherien said heterologous loop domain comprises at least 4 consecutive amino acids of a factor D loop domain.

3. The polypeptide of claim 1, wherein said heterologous loop domain comprises at least 10 consecutive amino acids of a factor D loop domain.

4. The polypeptide of claim 1, wherein said polypeptide comprises a heterologous loop domain sequence in microplasmin loop 3.

5. The polypeptide of claim 1, wherein said polypeptide comprises amino acid sequence LNGA (SEQ ID NO:1) in microplasmin loop 3.

6. The polypeptide of claim 1, wherein said polypeptide comprises a heterologous loop domain sequence in microplasmin loop 5.

7. The polypeptide of claim 1, wherein said polypeptide comprises amino acid sequence AHCLEDAADGKV (SEQ ID NO:2) in microplasmin loop 5.

8. The polypeptide of claim 1, wherein said polypeptide comprises a heterologous loop domain sequence in microplasmin loop 6.

9. The polypeptide of claim 1, wherein said polypeptide comprises amino acid sequence AHSLSQPEPSK (SEQ ID NO:3) in microplasmin loop 6.

10. The polypeptide of claim 1, wherein said polypeptide comprises a heterologous loop domain sequence in microplasmin loop 7.

11. The polypeptide of claim 1, wherein said polypeptide comprises amino acid sequence HPDSQPDTIDHD (SEQ ID NO:4) in microplasmin loop 7.

12. A method of dissolving a blood clot, comprising contacting said blood clot with the polypeptide of claim 1.

13. A substantially pure fragment of plasminogen, wherein said fragment is activated at least 10% more efficiently compared to human glu-plasminogen.

14. The fragment of claim 13, wherein said fragment comprises at least 150 consecutive residues of SEQ ID NO:17.

15. The fragment of claim 13, wherein said fragment comprises a methionine residue at the N-terminal end.

16. A substantially pure polypeptide comprising residues 550-810 of SEQ ID NO:17, wherein residue 555 is not a cysteine residue.

17. A substantially pure polypeptide comprising residues 550-810 of SEQ ID NO:17, wherein residue 560 is not a cysteine residue.

18. A substantially pure polypeptide comprising residues 550-810 of SEQ ID NO:17, wherein residue 580 is not an arginine residue.

19. A substantially pure polypeptide comprising residues 481-810 of SEQ ID NO:17, wherein residue 555 is not a cysteine residue or wherein residue 560 is not a cysteine residue.

20. A substantially pure polypeptide comprising residues 481-810 of SEQ ID NO:17, wherein residue 580 is not an arginine residue.
